(19) United States
(12) Patent Application Publication
(10) Pub. No.: US 2025/0152662 A1
(43) Pub. Date: May 15, 2025

(54) METHODS OF TREATING A CYTOKINE STORM RELATED DISEASE WITH RECOMBINANT MUTATED HUMAN ANGIOPOIETIN-LIKE 4

(71) Applicant: Sumant Singh CHUGH, Chicago, IL (US)

(72) Inventor: Sumant Singh CHUGH, Chicago, IL (US)

(73) Assignee: RUSH UNIVERSITY MEDICAL CENTER, Chicago, IL (US)

(21) Appl. No.: 18/838,145

(22) PCT Filed: Feb. 13, 2023
(86) PCT No.: PCT/US2023/062503

§ 371 Date: Aug. 13, 2024

Related U.S. Application Data
(60) Provisional application No. 63/309,754, filed on Feb. 14, 2022.

(51) Int. Cl. A61K 38/17

(52) U.S. Cl. CPC A61K 38/17

(57) ABSTRACT

The present disclosure provides novel approaches to the prevention, treatment, and amelioration of a disease in a patient wherein the disease is preceded, induced or exacerbated by a cytokine storm. More particularly, the present disclosure describes the administration of Angiopoietin-like 4 protein (ANGPTL4) to inhibit, prevent or neutralize the biological effects of certain cytokines and related proteins. Also contemplated herein are kits and compositions configured for use in the inhibition, prevention or neutralization of the biological effects of certain cytokines and related proteins.

### RELATED APPLICATIONS

This application claims priority to U.S. Provisional Patent Application No. 63/309,754 filed on Feb. 14, 2022, titled “Methods of Treating a Cytokine Storm Related Disease with Recombinant Mutated Human Angiopoietin-Like 4”, the entire content of which is incorporated herein.

### GOVERNMENT FUNDING

This invention was made with government support under several grants awarded by National Institutes of Health including R01DK101637, R01DK128203 and R01DK129522. The government has certain rights.

### BACKGROUND

Cytokine storms may complicate many types of human diseases. Viral infections, for example, can trigger cytokine production as part of the innate and adaptive immune response. The extensive cytokine storm documented early in the COVID-19 pandemic may be involved in organ damage and developed novel evidence-based models of cytokine mediated end organ damage. See Huang et al., “Clinical features of patients infected with 2019 novel coronavirus in Wuhan, China,” (2020) Lancet 395: pp. 497-506. Of the three organs studied, the literature on cardiac involvement shows elevated cardiac Troponin I levels (that mimic an acute myocardial infarction), myocarditis, myocardial necrosis, pericarditis, arrythmias and heart failure. See Topol, “COVID-19 can affect the heart,” (2020) Science 370: pp. 408-409; Inciardi et al., “Cardiac involvement in a patient with coronavirus disease 2019 (COVID-19),” (2020) JAMA Cardiol. 5: pp. 819-824. Evidence of liver injury include increased aminotransferase levels, hepatocyte injury, inflammation and steatosis. See Herta et al., “COVID-19 and the liver—Lessons learned,” (2021) Liver Int. 41 Suppl 1: pp. 1-8. Kidney manifestations are very common in hospitalized COVID-19 patients, with nearly 40% developing proteinuria, and about one-third developing acute kidney injury (AKI). See Cheng et al., “Kidney disease is associated with in-hospital death of patients with COVID-19,” (2020) Kidney Int. 97: pp. 829-838; Hirsch et al., “Acute kidney injury in patients hospitalized with COVID-19,” (2020) Kidney Int. 98: pp. 209-218. Kidney biopsy studies in COVID-19 patients with severe proteinuria and/or kidney dysfunction have most commonly documented the collapsing variant of focal and segmental glomerulosclerosis (FSGS) and acute kidney injury. See Kudose et al., “Kidney Biopsy Findings in Patients with COVID-19,” (2020) J Am Soc Nephrol. 31: pp. 1959-1968; Nasr et al., “Kidney Biopsy Findings in Patients With COVID-19, Kidney Injury and Proteinuria,” (2021) Am J Kidney Dis. 77: pp. 465-468 (2021).

The COVID-19 cytokine storm model, with respect to kidney disease, is a potential mechanistic comparison with rare manifestations of a common cold cytokine storm, with which it shares some components. See Basnet et al., “Rhinoviruses and Their Receptors,” (2019) Chest 155: pp. 1018-1025; Wine et al., “Cytokine responses in the common cold and otitis media,” (2012) Curr Allergy Asthma Rep. 12: pp. 574-581; Nieters et al., “Cross-sectional study on cytokine polymorphisms, cytokine production after T-cell stimulation and clinical parameters in a random sample of a German population,” (2001) Hum Genet. 108: pp. 241-248; Noah et al., “Nasal cytokine production in viral acute upper respiratory infection of childhood,” (1995) J Infect Dis. 171: pp. 584-592; van Kempen et al., “An update on the pathophysiology of rhinovirus upper respiratory tract infections,” (1999) Rhinology 37: pp. 97-103; Whiteman et al., “IFN-gamma regulation of ICAM-1 receptors in bronchial epithelial cells: soluble ICAM-1 release inhibits human rhinovirus infection,” (2008) J Inflamm (Lond). 5: p. 8; Jartti et al., “Systemic T-helper and T-regulatory cell type cytokine responses in rhinovirus vs. respiratory syncytial virus induced early wheezing: an observational study,” (2009) Respir Res. 10: p. 85; Hershey et al., “The association of atopy with a gain-of-function mutation in the alpha subunit of the interleukin-4 receptor,” (1997) N Engl J Med. 337: pp. 1720-1725; Abdel-Hafez et al., “Idiopathic nephrotic syndrome and atopy: is there a common link?,” (2009) Am J Kidney Dis. 54: pp. 945-953.

Common colds, frequently caused by Rhinoviruses, trigger nearly 70% of episodes of relapse of the glomerular diseases MCD and FSGS. See Passioti et al., “The common cold: potential for future prevention or cure,” (2014) Curr Allergy Asthma Rep. 14: p. 413; Takahashi et al., “Triggers of relapse in steroid-dependent and frequently relapsing nephrotic syndrome,” (2007) Pediatr Nephrol. 22: pp. 232-236.

Whereas this relapse pathway is unknown, the involvement of a cytokine storm remains unclear. Despite efforts to understand the resultant cytokine storm seen following COVID-19 infection, there is still a need to understand the underlying mechanisms of this phenomenon and the resultant clinical manifestations. Such an understanding will facilitate the design of therapeutic approaches to reduce cytokine storm related organ damage.

### SUMMARY OF THE DISCLOSURE

The present disclosure relates to a method for preventing or treating the effects of a disease in a patient comprising the step of administering to the patient a pharmaceutical composition comprising human Angiopoietin-like 4 protein (ANGPTL4), wherein the ANGPTL4 neutralizes the biological effects of one or more cytokines from the patient and wherein the disease is preceded, induced or exacerbated by a cytokine storm. The human Angiopoietin-like 4 protein (ANGPTL4) can be a recombinant form of human Angiopoietin-like 4 protein (ANGPTL4). In certain embodiments, the human Angiopoietin-like 4 protein (ANGPTL4) is a mutated recombinant form of human Angiopoietin-like 4 protein (ANGPTL4). In certain embodiments, the human Angiopoietin-like 4 protein (ANGPTL4) is protein 8520 (SEQ ID NO. 5), protein 8496 (SEQ ID NO. 6), protein 8501 (SEQ ID NO. 7), protein 8506 (SEQ ID NO. 8), protein 8511 (SEQ ID NO. 9), or protein 8515 (SEQ ID NO. 10). In certain embodiments, the ANGPTL4 is a polypeptide having an amino acid sequence with at least 90% sequence identity to SEQ ID NO: 5 (protein 8520).

In certain embodiments, the human Angiopoietin-like 4 protein (ANGPTL4) is capable of inhibiting the biological effects of one or more cytokines and related proteins in the patient. The one or more cytokines and related proteins are selected from the group comprising IL-2, IL-4, soluble IL-4Rα, IL-6, IL-13, IFN-γ, TNFα, soluble ICAM-1, and ACE-2.

In certain embodiments of the method, the cytokine storm is the result of a viral infection. In some embodiments, the viral infection is caused by an infection selected from the group consisting of SARS-CoV-1, SARS-CoV-2, Rhinoviruses, influenza, parainfluenza, Respiratory Syncytial Virus, adenoviruses, enteroviruses, other coronaviruses, cyto-megalovirus (CMV), Epstein Barr Virus (EBV), Middle East Respiratory Syndrome (MERS), and Ebola virus.

In certain embodiments of the method, the disease is induced by sepsis, primary or secondary hemophagocytic lymphohistiocytosis (HLH), an autoinflammatory disorder, group A streptococcus, bacteria, fungi, a tumor or other cancer, organ transplantation, diabetes mellitus, or metabolic syndrome.

In certain embodiments, the disease is a viral disease. In certain embodiments, the viral disease is SARS-CoV-1, SARS-CoV-2, Rhinoviruses, influenza, parainfluenza, Respiratory Syncytial Virus, adenoviruses, enteroviruses, other coronaviruses, cytomegalovirus (CMV), Epstein Barr Virus (EBV), Middle East Respiratory Syndrome (MERS), or Ebola virus.

In certain embodiments, the disease affects the heart, liver and kidney following the development or worsening of a cytokine storm. In other embodiments these phenomena are acute kidney injury, myocarditis, pericarditis, cardiac injury, hepatitis and liver injury, and worsening of glomerular diseases like Minimal Change Disease (MCD) and Focal and Segmental Glomerulosclerosis (FSGS).

Also disclosed herein is a method for inhibiting acute relapse or worsening of glomerular disease in a patient, wherein the disease is preceded, induced or exacerbated by a cytokine storm, comprising administration to the patient of a pharmaceutical composition comprising human Angiopoietin-like 4 protein (ANGPTL4), wherein the ANGPTL4 inhibits or neutralizes the biological effects of one or more cytokines in the subject. In certain embodiments, the human Angiopoietin-like 4 protein (ANGPTL4) is a recombinant form of human Angiopoietin-like 4 protein (ANGPTL4). In certain embodiments, the human Angiopoietin-like 4 protein (ANGPTL4) is a mutated recombinant form of human Angiopoietin-like 4 protein (ANGPTL4). In certain embodiments, the mutated recombinant form of human Angiopoietin-like 4 protein (ANGPTL4) is selected from the group consisting of: protein 8520 (SEQ ID NO. 5), protein 8496 (SEQ ID NO. 6), protein 8501 (SEQ ID NO. 7), protein 8506 (SEQ ID NO. 8), protein 8511 (SEQ ID NO. 9), and protein 8515 (SEQ ID NO. 10). In certain embodiments, the ANGPTL is a polypeptide with at least 90% sequence identity with SEQ ID NO. 5. In certain embodiments, the human Angiopoietin-like 4 protein (ANGPTL4) is capable of inhibiting the effects of circulating levels of one or more cytokines and related proteins in the patient.

In certain embodiments, the glomerular disease comprises nephrotic syndrome. In certain embodiments, the glomerular disease comprises minimal change disease, focal segmented glomerulosclerosis, membranous nephropathy, membranoproliferative glomerulonephritis, diabetic nephropathy, or lupus nephritis. In certain embodiments, the glomerular disease comprises acute relapse or worsening of chronic kidney disease caused by minimal change disease, focal segmented glomerulosclerosis, membranous nephropathy, membranoproliferative glomerulonephritis, diabetic nephropathy, or lupus nephritis.

In certain embodiments, the patient is in complete or partial remission of glomerular disease prior to the cytokine storm. In certain embodiments, the cytokine storm is induced by a viral infection. In certain embodiments, the viral infection comprises rhinovirus, human coronavirus, adenovirus, respiratory syncytial virus, enteroviruses other than rhinoviruses, parainfluenza virus, metapneumovirus, influenza, or combination thereof. In certain embodiments, administration occurs within about 1 hour to about 7 days of onset of the cytokine storm. In certain embodiments, administration occurs no later than about 7 days after of onset of the cytokine storm. In certain embodiments, the administration comprises parenteral administration. In certain embodiments, the administration comprises intravenous, intraarterial, intramuscular, intradermal, subcutaneous, or intraperitoneal administration. In certain embodiments, the administration comprises intravenous administration.

### BRIEF DESCRIPTION OF THE DRAWINGS

The following detailed description of the present disclosure may be better understood, by way of example only, with reference to the following drawings.

FIGS. 1A-1I show the effect of cytokine storms in a COVID-19 model. FIG. 1A is a flow chart depicting a cytokine storm model in a COVID-19 model resulting in multi-system organ injury. FIG. 1B is a chart listing the cytokine components of five different cocktails intended to mimic either the common cold or coronavirus. FIG. 1C is a chart that shows that baseline (i.e., day 0) urine albumin to creatinine ratio was similar between BALB/c mice treated with ANGPTL4 or control (human albumin), whereas at 24 hours post-treatment, the urine albumin to creatinine ratio was significantly lower in the ANGPTL4-treated group compared to the control-treated group (P<0.05). FIG. 1D show electron microscopy images demonstrating morphological changes in kidney glomeruli and tubules that were significantly improved in the ANGPTL4-treated mice compared to human albumin-treated controls. FIG. 1E is a table listing the histological changes in kidney glomeruli and tubules in the ANGPTL4-treated mice compared to human albumin-treated controls. Histological changes were significantly improved in the ANGPTL4 treated mice. FIG. 1F are histological images of the heart in ANGPTL4-treated BALB/C mice compared to human albumin-treated controls. Histological changes were significantly improved in the ANGPTL4 treated mice. FIG. 1G is a chart listing the morphological changes in heart tissue in the ANGPTL4-treated mice compared to human albumin-treated controls. Morphological changes were significantly improved in the ANGPTL4 treated mice. FIG. 1H are histological images of the liver in ANGPTL4-treated BALB/c mice compared to human albumin-treated controls. Histological changes were significantly improved in the ANGPTL4 treated mice. FIG. 1I is a Table listing the morphological changes in the liver in the ANGPTL4-treated mice compared to human albumin-treated controls. Morphological changes were significantly improved in the ANGPTL4 treated mice.

FIGS. 2A-2D show the effect of cytokine storms in primary glomerular diseases like Minimal Change Disease (MCD) and Focal and Segmental Glomerulosclerosis (FSGS). FIG. 2A is a flow chart depicting a cytokine storm model in a common cold model resulting in multi-system organ injury. FIG. 2B is a chart that shows that baseline (i.e., day 0) urine albuminuria was similar between BALB/cJ mice treated with ANGPTL4 or control (human albumin), whereas at 24 hours post-treatment, the urine albuminuria did not increase significantly in the ANGPTL4-treated group, but did rise significantly in the control-treated group (P<0.01). FIG. 2C shows electron microscopy images demonstrating that glomerular morphology was significantly better preserved in ANGPTL4-treated BALB/cJ mice compared to the human albumin-treated control group. FIG. 2D is a chart listing the changes in glomerular morphology in ANGPTL4-treated BALB/cJ mice compared to the human albumin-treated control group. Morphological changes were significantly improved in the ANGPTL4 treated mice.

### DETAILED DESCRIPTION

Herein, the applicants propose the use of proteins that improve and protect the vascular integrity in organs so as to prevent leakage of circulating proteins and inflammatory cells that can cause target organ damage. Applicants have identified Angiopoietin-like 4 (ANGPTL4) (wild type and mutated human protein) as having potent anti-apoptotic effects that may help to maintain vascular integrity by potent anti-apoptotic effects, thereby preventing leakage of circulating pro-injury proteins and cells. In areas of existing damage, ANGPTL4 may keep repair vessels open so as to promote healing. The recombinant human mutated ANGPTL4 has many advantages over wild type protein, including a longer half-life, no induction of hypertriglyceridemia, and lack of side effects that can be associated with ANGPTL4 fragments.

Applicants herein propose the administration of recombinant mutated human ANGPTL4 (e.g., protein 8520 and other previously known mutants) to treat cytokine storm related diseases like viral infections (SARS-CoV-2/COVID-19, common cold and other respiratory viruses), numerous viruses, bacteria, fungi, other micro-organisms that cause human illnesses, tumors and cancers that release cytokines, and numerous other human conditions associated with cytokine release.

The present disclosure provides novel approaches to the prevention, treatment, and amelioration of a disease in a patient wherein the disease is preceded, induced or exacerbated by a cytokine storm. More particularly, the present disclosure describes the administration of Angiopoietin-like 4 protein (ANGPTL4) to inhibit, prevent or neutralize the biological effects of certain cytokines and related proteins. Also contemplated herein are kits and compositions configured for use in the inhibition, prevention or neutralization of the biological effects of certain cytokines and related proteins.

Throughout this disclosure, various quantities, such as amounts, sizes, dimensions, proportions and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

As used herein, unless specifically stated or obvious from context, the term “about” in reference to a number or range of numbers is understood to mean the stated number and numbers +/−10% thereof, or 10% below the lower listed limit and 10% above the higher listed limit for the values listed for a range.

As used herein, the term “patient” refers to a human.

As used herein, the term “inhibit” means to prevent, delay, and/or to reduce the effect or likelihood of something happening.

As used herein, the term “pharmaceutically acceptable derivative” means any pharmaceutically acceptable salt, ester, salt of an ester, solvate or other derivative of an ANGPTL4 polypeptide or polypeptide derivative of the present disclosure that, upon administration to a subject, is capable of providing (directly or indirectly) the function of wild type ANGPTL4; in certain embodiments, the ANGPTL4 polypeptide or polypeptide derivative shows decreased LPL inhibitory activity of a resistance to cleavage. Particularly favored derivatives are those that increase the bioavailability of an ANGPTL4 polypeptide or polypeptide derivative of the disclosure when such polypeptides are administered to a subject (e.g., by allowing an orally administered compound to be more readily absorbed into the blood), enhance delivery of such polypeptides to a given biological compartment, increase solubility to allow administration by injection, alter metabolism or alter rate of excretion. In one embodiment, the derivative is a prodrug.

As used herein, the term “pharmaceutically acceptable salt(s)”, unless otherwise indicated, includes salts of acidic or basic groups that may be present in the ANGPTL4 polypeptide or polypeptide derivative of the present disclosure.

As used herein, the terms “preventing” or “to prevent” refers to minimizing, reducing or suppressing the risk of developing a disease state or parameters relating to the disease state or progression or other abnormal or deleterious conditions.

As used herein, the term “related proteins” with respect to cytokines means proteins that are induced by, capable of binding to, or otherwise associated with the release of cytokines. By way of example, angiotensin-converting enzyme 2 (ACE-2), to which SARS-CoV-2 binds on cell surfaces resulting in cytokine release, is one such “related protein” as contemplated by this disclosure.

As used herein, the term “sequence identity”, unless otherwise indicated, means a comparison between pairs of nucleic acid or amino acid molecules, i.e., the relatedness between two amino acid sequences or between two nucleotide sequences. In general, the sequences are aligned so that the highest order match is obtained. Methods for determining sequence identity are known and can be determined by commercially available computer programs that can calculate % identity between two or more sequences. A typical example of such a computer program is CLUSTAL. As an illustration, by a polynucleotide having a nucleotide sequence having at least, for example, 90% sequence identity to a reference nucleotide sequence is intended that the nucleotide sequence of the polynucleotide is identical to the reference sequence except that the polynucleotide sequence may include on average of up to 10 point mutations per each 100 nucleotides of the reference nucleotide sequence. In other words, to obtain a polynucleotide having a nucleotide sequence at least 90% identical to a reference nucleotide sequence, up to 10% of the nucleotides in the reference sequence may be deleted or substituted with another nucleotide, or a number of nucleotides up to 10% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Similarly, by a polypeptide having an amino acid sequence having at least, for example, 90% sequence identity to a reference amino acid sequence is intended that the amino acid sequence of the polypeptide is identical to the reference sequence except that the polypeptide sequence may include on average up to 10 amino acid alterations per each 100 amino acids of the reference amino acid. In other words, to obtain a polypeptide having an amino acid sequence at least 90% identical to a reference amino acid sequence, up to 10% of the amino acid residues in the reference sequence may be deleted or substituted with another amino acid, or a number of amino acids up to 10% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As used herein, the term “therapeutically effective amount” refers to an amount of ANGPTL4, either alone or as a part of a pharmaceutical composition, that is capable of having any detectable, positive effect on any symptom, aspect, or characteristics of a disease or condition. Such effect need not be absolute to be beneficial.

As used herein, the terms “treating” or “to treat” includes restraining, slowing, stopping, or reversing the progression or severity of an existing symptom or disorder.

The term “ANGPTL4” refers herein to Human Angiopoietin-like 4. Exemplary forms of ANGPTL4 include, but are not limited to, mutated forms of Human Angiopoietin-like 4, such as protein 8520 (SEQ ID NO. 5), protein 8496 (SEQ ID NO. 6), protein 8510 (SEQ ID NO. 7), protein 8506 (SEQ ID NO. 8), protein 8511 (SEQ ID NO. 9), or protein 8515 (SEQ ID NO. 10). ANGPTL4 is a polypeptide that in humans is encoded by the ANGPTL4 gene. This gene is a member of the angiopoietin/angiopoietin-like gene family and encodes a glycosylated, secreted protein with a N-terminal signal sequence, a coiled-coil domain, a linker region and a fibrinogen C-terminal domain. This gene is induced under hypoxic conditions in endothelial cells and is the target of peroxisome proliferation activators. The encoded protein is a serum hormone directly involved in regulating glucose homeostasis, lipid metabolism, and insulin sensitivity and also acts as an apoptosis survival factor for vascular endothelial cells. Alternatively spliced transcript variants encoding different isoforms have been described. This gene was previously referred to as ANGPTL2 but has been renamed ANGPTL4.

### Cytokine Effect Inhibition

In any of the disclosed embodiments, the biological effects of one or more cytokines and related proteins to be inhibited or neutralized comprise IL-2, IL-4, soluble IL-4Rα, IL-6, IL-13, IFN-γ, TNFα, soluble ICAM-1, and ACE-2. It will be understood for the disclosure herein that depending upon the severity of the viral infection or other condition being treated, the inhibition or neutralization of the biological effects of more than one cytokine and/or related protein may be more effective than the inhibition or neutralization of the effects of a single cytokine and/or related protein.

The current invention contemplates that the biological effects of various cytokines and/or related proteins can be neutralized or inhibited by administration of ANGPTL4. For example, as described herein, the biological effects of cytokines and/or related proteins can be neutralized or inhibited by administration of a therapeutically effective amount of an agent where the agent comprises ANGPTL4.

The ANGPTL4 can be administered once per day, two or more times daily or once per week. The ANGPTL4 can be administered by any conventional means including orally, intramuscularly, intraperitoneally or intravenously into the subject. If injected, the ANGPTL4 can be injected at a single site per dose or multiple sites per dose. SEQ ID NOS. 1 and 2 show amino acid and cDNA sequence from human (Protein Variant 1 isoform a, long form), whereas SEQ ID NOS. 3 and 4 show amino acid and cDNA sequence from human (Protein Variant 3 isoform b, short form). SEQ ID NO. 5 shows the amino acid and nucleic acid substitutions for mutant human ANGPTL4 (protein 8520). SEQ ID NO. 6 shows the amino acid and nucleic acid substitutions for mutant human ANGPTL4 (protein 8496). SEQ ID NO. 7 shows the amino acid and nucleic acid substitutions for mutant human ANGPTL4 (protein 8501). SEQ ID NO. 8 shows the amino acid and nucleic acid substitutions for mutant human ANGPTL4 (protein 8506). SEQ ID NO. 9 shows the amino acid and nucleic acid substitutions for mutant human ANGPTL4 (protein 8511). SEQ ID NO. 10 shows the amino acid and nucleic acid substitutions for mutant human ANGPTL4 (protein 8515).

### Compositions

Useful compositions of the present disclosure may comprise ANGPTL4 as described herein that is useful in the treatment and prevention methods of the present disclosure. The compositions disclosed may comprise one or more of such compounds, in combination with a pharmaceutically acceptable carrier. Examples of such carriers and methods of formulation may be found in Remington: The Science and Practice of Pharmacy (20th Ed., Lippincott, Williams & Wilkins, Daniel Limmer, editor) and are known to those of skill in the art. To form a pharmaceutically acceptable composition suitable for administration, such compositions will contain an therapeutically effective amount of compound.

The pharmaceutical compositions of the disclosure may be used in the treatment and prevention methods of the present disclosure. Such compositions are administered to a subject in amounts sufficient to deliver a therapeutically effective amount of the compound(s) so as to be effective in the treatment and prevention methods disclosed herein. The therapeutically effective amount may vary according to a variety of factors such as, but not limited to, the subject's condition, weight, sex and age. Other factors include the mode and site of administration. The pharmaceutical compositions may be provided to the subject in any method known in the art. Exemplary routes of administration include, but are not limited to, subcutaneous, intravenous, topical, epicutaneous, oral, intraosseous, and intramuscular. The compositions of the present disclosure may be administered only one time to the subject or more than one time to the subject. Furthermore, when the compositions are administered to the subject more than once, a variety of regimens may be used, such as, but not limited to, one per day, once per week or once per month. The compositions may also be administered to the subject more than one time per day. The therapeutically effective amount and appropriate dosing regimens may be identified by routine testing in order to obtain optimal activity, while minimizing any potential side effects. In addition, co-administration or sequential administration of other agents may be desirable.

The compositions of the present disclosure may be administered systemically, such as by intravenous administration, or locally such as by subcutaneous injection or by application of a paste or cream.

In one embodiment, a nucleic acid, which may be in the form of a suitable plasmid or vector, is provided that codes for an ANGPTL4 polypeptide or ANGPTL4 polypeptide variant of the present disclosure. Such nucleic acid is introduced into a cell, which may be obtained from the subject, by suitable methods known in the art (for example, electroporation). In one embodiment, the cell is an adipose cell. The cells may be assayed for expression of the ANGPTL4 polypeptide or polypeptide derivative (in one embodiment, expression of the polypeptide can be determined by the presence of a tag on the polypeptide as discussed herein). The cells expressing an ANGPTL4 polypeptide of polypeptide derivative may then be introduced into the subject. In one embodiment, the cells are administered to the subject by subcutaneous injection; other methods of administration may also be used, including those discussed herein. The cells then express ANGPTL4 polypeptide or an ANGPTL4 polypeptide derivative, which is taken up into the circulation.

The compositions of the present disclosure may further comprise agents which improve the solubility, half-life, absorption, etc. of the compound(s). Furthermore, the compositions of the present disclosure may further comprise agents that attenuate undesirable side effects and/or or decrease the toxicity of the compounds(s). Examples of such agents are described in a variety of texts, such a, but not limited to, Remington: The Science and Practice of Pharmacy (20th Ed., Lippincott, Williams & Wilkins, Daniel Limmer, editor).

The compositions of the present disclosure can be administered in a wide variety of dosage forms for administration. For example, the compositions can be administered in forms, such as, but not limited to, tablets, capsules, sachets, lozenges, troches, pills, powders, granules, elixirs, tinctures, solutions, suspensions, elixirs, syrups, ointments, creams, pastes, emulsions, or solutions for intravenous administration or injection. Other dosage forms include administration transdermally, via patch mechanism or ointment. Any of the foregoing may be modified to provide for timed release and/or sustained release formulations.

In the present disclosure, the pharmaceutical compositions may further comprise a pharmaceutically acceptable carriers include, but are not limited to, vehicles, adjuvants, surfactants, suspending agents, emulsifying agents, inert fillers, diluents, excipients, wetting agents, binders, lubricants, buffering agents, disintegrating agents and carriers, as well as accessory agents, such as, but not limited to, coloring agents and flavoring agents (collectively referred to herein as a carrier). Typically, the pharmaceutically acceptable carrier is chemically inert to the active compounds and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, for oral administration in solid form, such as but not limited to, tablets, capsules, sachets, lozenges, troches, pills, powders, or granules, the compound(s) may be combined with an oral, non-toxic pharmaceutically acceptable inert carrier, such as, but not limited to, inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid as well as the other carriers described herein. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

For oral liquid forms, such as but not limited to, tinctures, solutions, suspensions, elixirs, syrups, the nucleic acid molecules of the present disclosure can be dissolved in diluents, such as water, saline, or alcohols. Furthermore, the oral liquid forms may comprise suitably flavored suspending or dispersing agents such as the synthetic and natural gums, for example, tragacanth, acacia, methylcellulose and the like. Moreover, when desired or necessary, suitable and coloring agents or other accessory agents can also be incorporated into the mixture. Other dispersing agents that may be employed include glycerin and the like.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound(s) may be administered in a physiologically acceptable diluent, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly (ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as, but not limited to, a soap, an oil or a detergent, suspending agent, such as, but not limited to, pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkylbeta-aminopropionates, and 2-alkylimidazoline quaternary ammonium salts, and (e) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight.

Topical dosage forms, such as, but not limited to, ointments, creams, pastes, emulsions, containing the nucleic acid molecule of the present disclosure, can be admixed with a variety of carrier materials well known in the art, such as, e.g., alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations may be applied to a patch, bandage or dressing for transdermal delivery or may be applied to a bandage or dressing for delivery directly to the site of a wound or cutaneous injury.

The compound(s) of the present disclosure can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. Such liposomes may also contain monoclonal antibodies to direct delivery of the liposome to a particular cell type or group of cell types.

The compound(s) of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyl-eneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

Some embodiments of administering the ANGPTL4 polypeptide or derivative to a patient involve a form of administration that delivers the polypeptide to the patient's blood. In one example the polypeptide is administered intravenously. Given the appropriate dosage form, such administration may be performed orally, subcutaneously, or by other means as is known in the art. The ANGPTL4 polypeptide or derivative may be administered in a therapeutically effective amount; this amount will generally be within a certain range of ratios of mass of compound to mass of subject. In some embodiments of the method the polypeptide is administered at a dosage of about 0.005-150,000 μg/kg, 0.5-15,000 μg/kg, 5-1500 μg/kg, or 50-150 μg/kg. Thus, for a typical 70 kg human adult, the dosage may be 0.0035-11,000 mg, 0.035-1100 mg, 0.35-110 mg, or 3.5-11 mg. Administration may occur on a regular schedule. In some embodiments of the method the polypeptide is administered about once per 14 days. In other embodiments the polypeptide is administered about twice per month. In still other embodiments the polypeptide is administered from about once per month to about twice per month. In further embodiments, the polypeptide is administered once per a given time period selected from the group consisting of: a day, two days, three days, a week, ten days, two weeks, three weeks, four weeks, and a month.

### EXAMPLES

The following examples are provided for the purpose of illustrating various embodiments of the invention and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art. In particular, the following studies illustrate the use of a recombinant mutated form of human Angiopoietin-like 4 protein (ANGPTL4) to improve disease outcomes in two different models associated with cytokine storms.

### Example 1

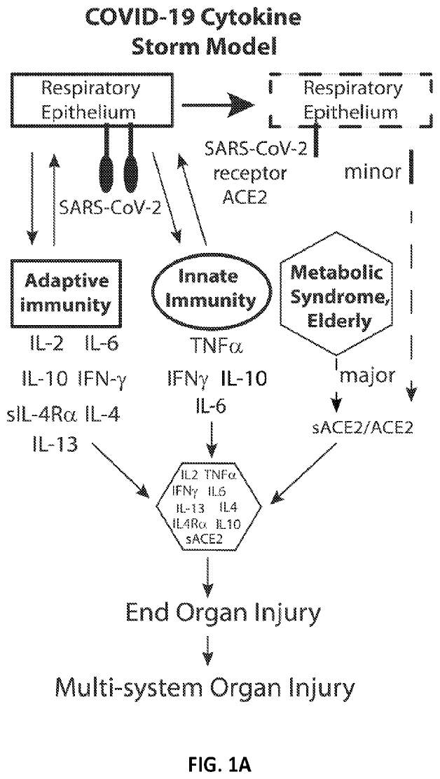

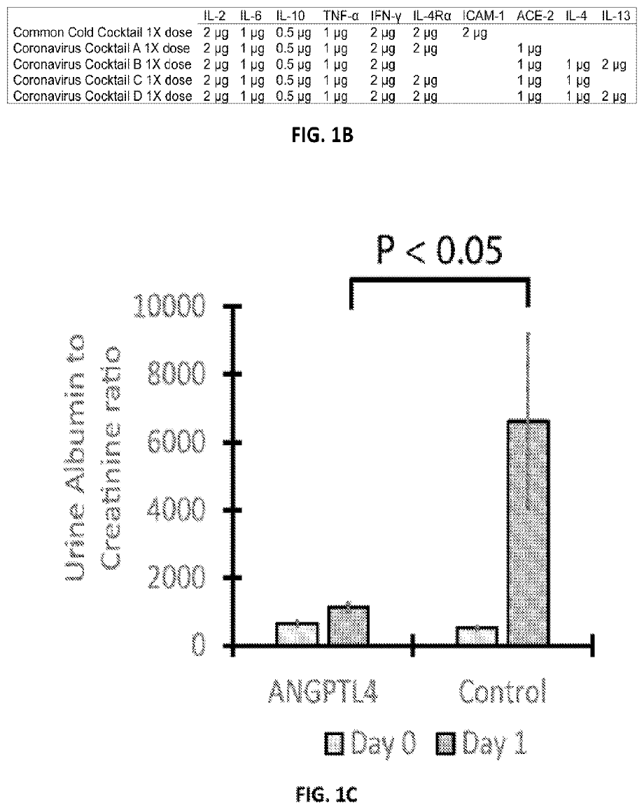

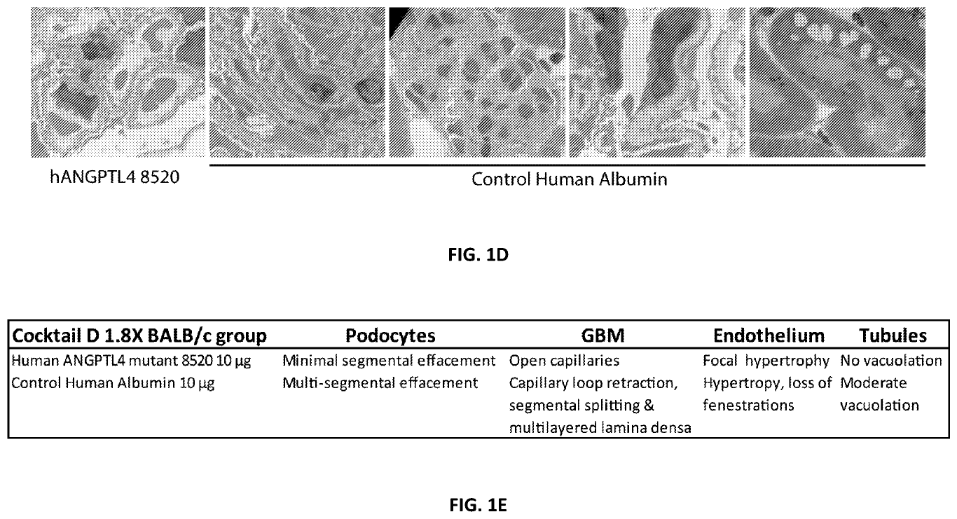

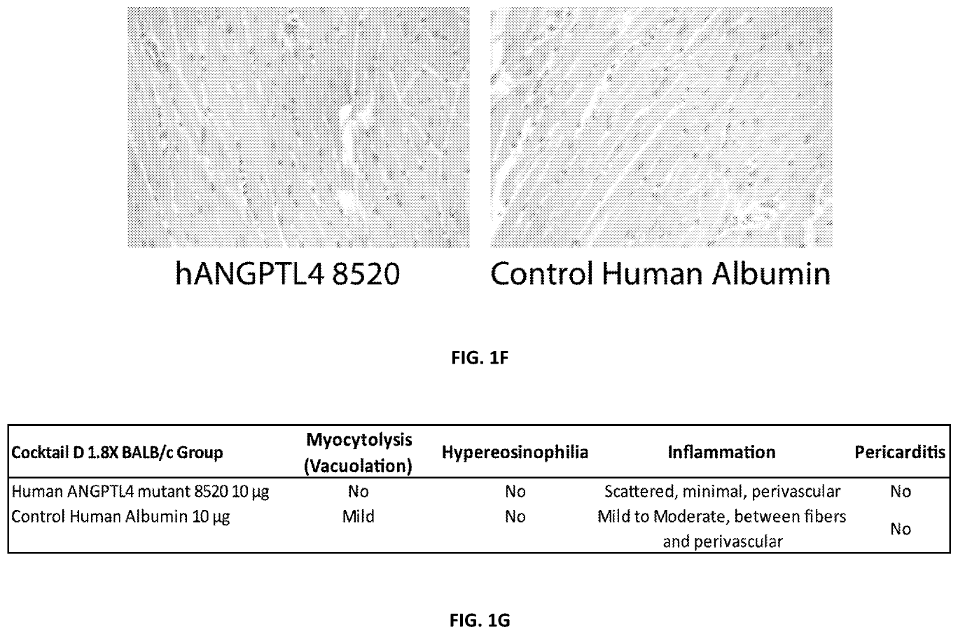

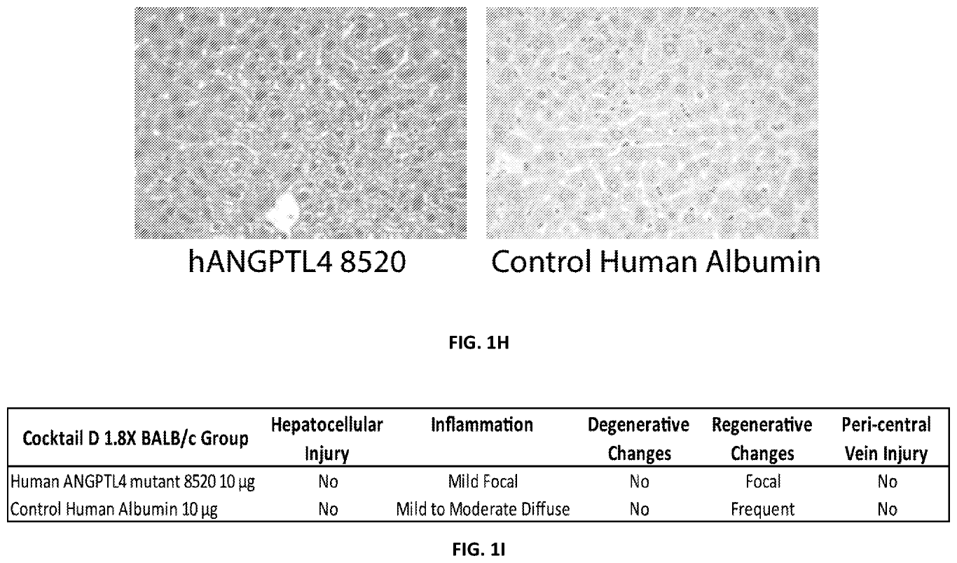

Ongoing studies in COVID-19 patients show that viral particles are not detectable in many organs affected in this disease. See refs 1-5. This raises the possibility that these organs may be affected by the extensive cytokine storm documented early on in the pandemic in hospitalized COVID-19 patients (FIG. 1A). Mutated forms of Human Angiopoietin-like 4 (protein 8520) were previously shown to reduce proteinuria in rat models of FSGS and diabetic nephropathy. See refs 6, 7. Using different components or innate and adaptive immunity triggered during infection, applicants developed cytokine cocktails that mimic the COVID-19 cytokine storm (FIG. 1B), and tested the ability of protein 8520 to reduce glomerular injury in this model. After baseline urine and blood collections, two groups of BALB/c mice (mean n=5 mice per group) were injected intravenously with dose 1.8× of COVID Cocktail D. One hour later, they received 10 μg of His-Tag purified protein 8520 or control human albumin. At 24 hours after model induction, blood and urine samples were collected, and the mice euthanized, followed by multiple organ harvesting and histological analysis. Urine albumin was measured by ELISA (Bethyl Labs), and urine and serum creatinine by Mass Spectrometry. FIG. 1C shows that baseline urine albumin to creatinine ratio was similar between the two groups. At 24 hours, urine albumin to creatinine ratio was significantly lower in the 8520 treated compared to control treated group (P<0.05). Serum creatinine on Day 1 was similar between both groups, and not significantly elevated compared to baseline. FIGS. 1D and 1E show electron microscopy morphological changes in kidney glomeruli and tubules, that were significantly improved in the Study (ANGPTL4 8520) group. FIGS. 1F and 1G show histological changes in the heart that were significantly improved in the ANGPTL4-treated group compared to controls. FIGS. 1H and 1I show histological changes in the liver, that were significantly improved in the ANGPTL4-treated group compared to controls.

### Example 2

In nearly 70% of patients with primary glomerular diseases like Minimal Change Disease (MCD) and Focal and Segmental Glomerulosclerosis (FSGS), relapse is preceded by a common cold. See ref 8. In addition, most patients with MCD have a chronic atopic state. See ref 9. Previous studies (see ref 10) showed the importance of low podocyte ZHX2 expression in the pathogenesis of MCD and FSGS.

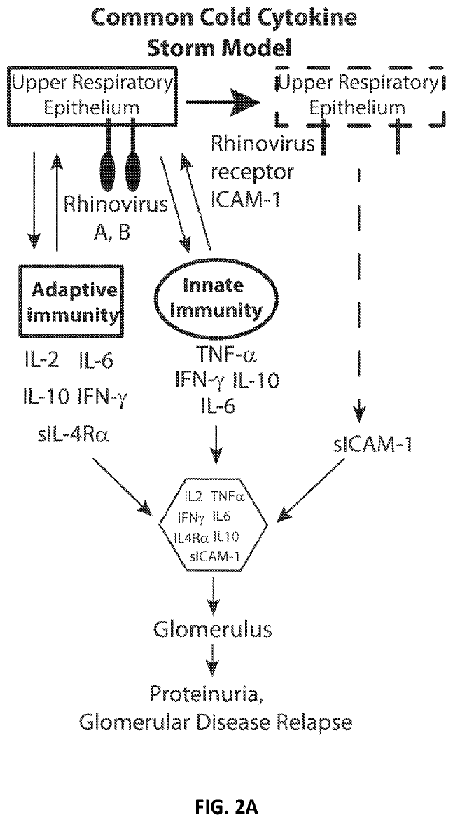

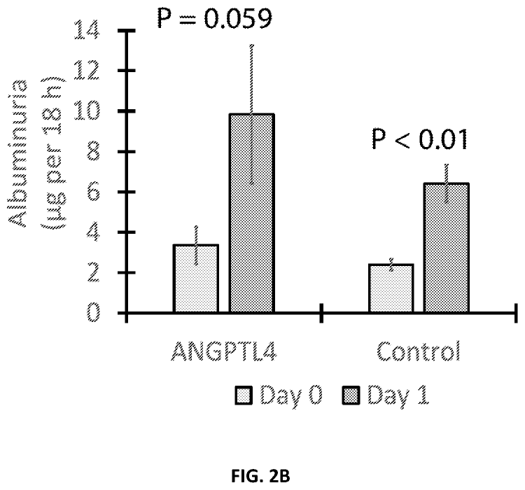

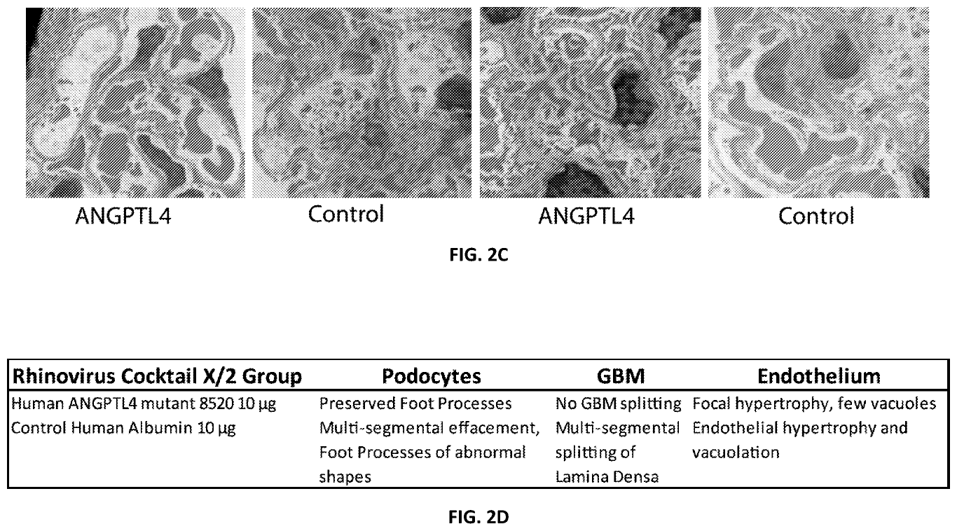

At least half of all common colds are caused by rhinovirus infections. The applicants developed a cytokine cocktail to mimic common cold induced relapse of MCD and FSGS (FIGS. 1B and 2A). When injected intravenously into BALB/cJ mice, the Rhinovirus Common Cold cocktail induced a dose-dependent increase in albuminuria to mimic a common cold induced relapse on glomerular disease. Applicants tested the ability of Mutated Human Angiopoietin-like 4 (protein 8520) (see refs 6, 7) to reduce albuminuria in this model of a mild cytokine storm. After baseline blood samples and 18-hour urine collections in metabolic cages, two groups of BALB/cJ mice were injected intravenously with dose X/2 of the Rhinovirus Common Cold cytokine cocktail. One hour later, they received 10 μg of His-Tag purified protein 8520 or control human albumin. At 24 hours after model induction, 18-hour urine samples were collected, blood samples taken and the mice euthanized, followed by harvesting of kidneys. Urine albumin was measured by ELISA (Bethyl Labs), and serum creatinine by Mass Spectrometry. Kidney sections were processed histology. FIG. 2B shows that baseline 18-hour urinary albumin excretion was similar between both groups. At 24 hours, urine albumin excretion increased significantly in the control human albumin treated group (P<0.01), but not in the 8520 treated group. All mice were normally active at 6 and 24 hours prior to euthanasia. FIGS. 2C and 2D show that glomerular morphology assessed by electron microscopy was significantly better preserved in ANGPTL4-treated BALB/cJ mice compared to the human albumin-treated control group.

### REFERENCES

1) Spudich S, Nath A. Nervous system consequences of COVID-19. Science. 2022 Jan. 21; 375(6578):267-269.
2) Gupta A, Madhavan M V, Sehgal K, Nair N, Mahajan S, Sehrawat T S, Bikdeli B, Ahluwalia N, Ausiello J C, Wan E Y, Freedberg D E, Kirtane A J, Parikh S A, Maurer M S, Nordvig A S, Accili D, Bathon J M, Mohan S, Bauer K A, Leon M B, Krumholz H M, Uriel N, Mehra M R, Elkind M S V, Stone G W, Schwartz A, Ho D D, Bilezikian J P, Landry D W. Extrapulmonary manifestations of COVID-19. Nat Med.2020 July; 26(7):1017-1032.
3) Lindner D, Fitzek A, Bräuninger H, Aleshcheva G, Edler C, Meissner K, Scherschel K, Kirchhof P, Escher F, Schultheiss H P, Blankenberg S, Püschel K, Westermann D. Association of Cardiac Infection With SARS-CoV-2 in Confirmed COVID-19 Autopsy Cases. JAMA Cardiol. 2020 Nov. 1; 5(11):1281-1285.
4) Hirsch J S, Ng J H, Ross D W, Sharma P, Shah H H, Barnett R L, Hazzan A D, Fishbane S, Jhaveri K D; Northwell COVID-19 Research Consortium; Northwell Nephrology COVID-19 Research Consortium. Acute kidney injury in patients hospitalized with COVID-19. Kidney Int. 2020 July; 98(1):209-218.
5) Kudose S, Batal I, Santoriello D, Xu K, Barasch J, Peleg Y, Canetta P, Ratner L E, Marasa M, Gharavi A G, Stokes M B, Markowitz G S, D'Agati V D. Kidney Biopsy Findings in Patients with COVID-19. J Am Soc Nephrol. 2020 September; 31(9):1959-1968.
6) Clement, L. C. et al. Circulating Angiopoietin-like 4 links proteinuria with hypertriglyceridemia in nephrotic syndrome. Nat. Med. 20, 37-46 (2014).
7) U.S. Pat. No. 10,023,620.
8) Takahashi, S. et al. Triggers of relapse in steroid-dependent and frequently relapsing nephrotic syndrome. Pediatr Nephrol. 22, 232-236 (2007).
9) Abd el-Hafez M, Shimada M, Lee P Y, Johnson R J, Garin E H. Idiopathic nephrotic syndrome and atopy: is there a common link? Am J Kidney Dis. 2009; 54(5):945-953.
10) Macé C, Del Nogal Avila M, Marshall C B, Donoro H, Kharlyngdoh J, Das R, Molina-Jijon E, Shastry S, Soria E, Wetzels J, Dijkman H, Avila-Casado C, Clement L C and Chugh S S. ZHX2 and its interacting proteins regulate upstream pathways in podocyte diseases. Kidney Int 2020 April; 97(4):753-764.

Although the present invention and its advantages have been described in detail, it should be understood that various changes, substitutions and alterations can be made herein without departing from the spirit and scope of the invention as defined in the appended claims. The methods, assays, and the various embodiments thereof described herein are exemplary. Various other embodiments of the methods and assays described herein are possible.

What is claimed:

1. A method for preventing a disease in a patient comprising the step of administering to the patient a pharmaceutical composition comprising human Angiopoietin-like 4 protein (ANGPTL4), wherein the ANGPTL4 inhibits or neutralizes the biological effects of one or more cytokines from the patient and wherein the disease is preceded, induced or exacerbated by a cytokine storm.

2. The method of any of claim 1, wherein the human Angiopoietin-like 4 protein (ANGPTL4) is a recombinant form of human Angiopoietin-like 4 protein (ANGPTL4).

3. The method of claim 1 wherein the human Angiopoietin-like 4 protein (ANGPTL4) is a mutated recombinant form of human Angiopoietin-like 4 protein (ANGPTL4).

4. The method of claim 3, wherein the mutated recombinant form of human Angiopoietin-like 4 protein (ANGPTL4) is selected from the group consisting of: protein 8520 (SEQ ID NO. 5), protein 8496 (SEQ ID NO. 6), protein 8501 (SEQ ID NO. 7), protein 8506 (SEQ ID NO. 8), protein 8511 (SEQ ID NO. 9), and protein 8515 (SEQ ID NO. 10).

5. The method of claim 3, wherein the ANGPTL is a polypeptide with at least 90% sequence identity with SEQ ID NO. 5.

6. The method of claim 1, wherein the human Angiopoietin-like 4 protein (ANGPTL4) is capable of inhibiting the effects of circulating levels of one or more cytokines and related proteins in the patient.

7. The method of claim 6, wherein the one or more cytokines and related proteins are selected from the group comprising IL-2, IL-4, soluble IL-4Rα, IL-6, IL-13, IFN-γ, TNFα, soluble ICAM-1, and ACE-2.

8. The method of claim 1, wherein the cytokine storm is the result of a viral infection.

9. The method of claim 8, wherein the viral infection is caused by an infection selected from the group consisting of SARS-CoV-1, SARS-CoV-2, rhinoviruses, influenza, parainfluenza, Respiratory Syncytial Virus, adenoviruses, enteroviruses, other coronaviruses, cytomegalovirus (CMV), Epstein Barr Virus (EBV), Middle East Respiratory Syndrome (MERS), and Ebola virus.

10. The method of claim 1, wherein the disease is induced by sepsis, primary or secondary hemophagocytic lymphohistiocytosis (HLH), an autoinflammatory disorder, group A streptococcus, bacteria, fungi, a tumor or other cancer, organ transplantation, diabetes mellitus, or metabolic syndrome.

11. The method of claim 1, wherein the disease is a viral disease.

12. The method of claim 11, wherein the viral disease is selected from the group consisting of SARS-CoV-1, SARS-CoV-2, rhinoviruses, influenza, parainfluenza, Respiratory Syncytial Virus, adenoviruses, enteroviruses, other coronaviruses, cytomegalovirus (CMV), Epstein Barr Virus (EBV), Middle East Respiratory Syndrome (MERS), and Ebola virus.

13. The method claim 1, wherein the disease is selected from the group comprising acute kidney injury, myocarditis, pericarditis, cardiac injury, and hepatitis and liver injury.

14. A method for treating a disease in a patient comprising the step of administering to the patient a pharmaceutical composition comprising human Angiopoietin-like 4 protein (ANGPTL4), wherein the ANGPTL4 inhibits or neutralizes the effects of one or more cytokines from the patient and wherein the disease is preceded, induced or exacerbated by a cytokine storm.

15. The method of any of claim 14, wherein the human Angiopoietin-like 4 protein (ANGPTL4) is a recombinant form of human Angiopoietin-like 4 protein (ANGPTL4).

16. The method of claim 14 wherein the human Angiopoietin-like 4 protein (ANGPTL4) is a mutated recombinant form of human Angiopoietin-like 4 protein (ANGPTL4).

17. The method of claim 16, wherein the mutated recombinant form of human Angiopoietin-like 4 protein (ANGPTL4) is selected from the group consisting of: protein 8520 (SEQ ID NO. 5), protein 8496 (SEQ ID NO. 6), protein 8501 (SEQ ID NO. 7), protein 8506 (SEQ ID NO. 8), protein 8511 (SEQ ID NO. 9), and protein 8515 (SEQ ID NO. 10).

18. The method of claim 16, wherein the ANGPTL is a polypeptide with at least 90% sequence identity with SEQ ID NO. 5.

19. The method of claim 14, wherein the human Angiopoietin-like 4 protein (ANGPTL4) is capable of inhibiting the effects of circulating levels of one or more cytokines and related proteins in the patient.

20. The method of claim 19, wherein the one or more cytokines and related proteins are selected from the group comprising IL-2, IL-4, soluble IL-4Rα, IL-6, IL-13, IFN-γ, TNFα, soluble ICAM-1, and ACE-2.

21. The method of claim 14, wherein the cytokine storm is the result of a viral infection.

22. The method of claim 21, wherein the viral infection is caused by an infection selected from the group consisting of SARS-CoV-1, SARS-CoV-2, rhinoviruses, influenza, parainfluenza, Respiratory Syncytial Virus, adenoviruses, enteroviruses, other coronaviruses, cytomegalovirus (CMV), Epstein Barr Virus (EBV), Middle East Respiratory Syndrome (MERS), and Ebola virus.

23. The method of claim 14, wherein the disease is induced by sepsis, primary or secondary hemophagocytic lymphohistiocytosis (HLH), an autoinflammatory disorder, group A streptococcus, bacteria, fungi, a tumor or other cancer, organ transplantation, diabetes mellitus, or metabolic syndrome.

24. The method of claim 14, wherein the disease is a viral disease.

25. The method of claim 24, wherein the viral disease is selected from the group consisting of SARS-CoV-1, SARS-CoV-2, influenza, parainfluenza, Respiratory Syncytial Virus, adenoviruses, enteroviruses, other coronaviruses, cytomegalovirus (CMV), Epstein Barr Virus (EBV), Middle East Respiratory Syndrome (MERS), and Ebola virus.

26. The method of claim 15, wherein the disease is selected from the group comprising acute kidney injury, myocarditis, pericarditis, cardiac injury, and hepatitis and liver injury.

27. A method for inhibiting acute relapse or worsening of glomerular disease in a patient, wherein the disease is preceded, induced or exacerbated by a cytokine storm, comprising administration to the patient of a pharmaceutical composition comprising human Angiopoietin-like 4 protein (ANGPTL4), wherein the ANGPTL4 inhibits or neutralizes the biological effects of one or more cytokines in the subject.

28. The method of any of claim 27, wherein the human Angiopoietin-like 4 protein (ANGPTL4) is a recombinant form of human Angiopoietin-like 4 protein (ANGPTL4).

29. The method of claim 27 wherein the human Angiopoietin-like 4 protein (ANGPTL4) is a mutated recombinant form of human Angiopoietin-like 4 protein (ANGPTL4).

30. The method of claim 29, wherein the mutated recombinant form of human Angiopoietin-like 4 protein (ANGPTL4) is selected from the group consisting of: protein 8520 (SEQ ID NO. 5), protein 8496 (SEQ ID NO. 6), protein 8501 (SEQ ID NO. 7), protein 8506 (SEQ ID NO. 8), protein 8511 (SEQ ID NO. 9), and protein 8515 (SEQ ID NO. 10).

31. The method of claim 29, wherein the ANGPTL is a polypeptide with at least 90% sequence identity with SEQ ID NO. 5.

32. The method of claim 27, wherein the human Angiopoietin-like 4 protein (ANGPTL4) is capable of inhibiting the effects of circulating levels of one or more cytokines and related proteins in the patient.

33. The method of claim 27, wherein the glomerular disease comprises nephrotic syndrome.

34. The method of claim 27, wherein the glomerular disease comprises minimal change disease, focal segmented glomerulosclerosis, membranous nephropathy, membranoproliferative glomerulonephritis, diabetic nephropathy, or lupus nephritis.

35. The method of claim 27, wherein the glomerular disease comprises acute relapse or worsening of chronic kidney disease caused by minimal change disease, focal segmented glomerulosclerosis, membranous nephropathy, membranoproliferative glomerulonephritis, diabetic nephropathy, or lupus nephritis.

36. The method of claim 27, wherein the patient is in complete or partial remission of glomerular disease prior to the cytokine storm.

37. The method of claim 27, wherein the cytokine storm is induced by a viral infection.

38. The method of claim 37, wherein the viral infection comprises rhinovirus, human coronavirus, adenovirus, respiratory syncytial virus, enteroviruses other than rhinoviruses, parainfluenza virus, metapneumovirus, influenza, or combination thereof.

39. The method of claim 27, wherein administration occurs within about 1 hour to about 7 days of onset of the cytokine storm.

40. The method of claim 27, wherein administration occurs no later than about 7 days after of onset of the cytokine storm.

41. The method of claim 27, wherein the administration comprises parenteral administration.

42. The method of claim 27, wherein the administration comprises intravenous, intraarterial, intramuscular, intradermal, subcutaneous, or intraperitoneal administration.

43. The method of claim 27, wherein the administration comprises intravenous administration.


SEQUENCE LISTING (10 sequences)

SEQ ID NO: 1 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 2 - DNA, 1967 bp, Homo sapiens
  source 1..1967: mol_type = genomic DNA
ataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacacttaaaaaaaaaaaaaaaaaaaaaaaaaaaaaaaaaaaaaaaaaaaaaaaaaaaaaaaaaaaaaaaa

SEQ ID NO: 3 - protein, 368 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHHGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 4 - DNA, 1853 bp, Homo sapiens
  source 1..1853: mol_type = genomic DNA
ataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacacttaaaaaaaaaaaaaaaaaaaaaaaaaaaaaaaaaaaaaaaaaaaaaaaaaaaaaaaaaaaaaaaa

SEQ ID NO: 5 - protein, 406 aa, synthetic construct
MSGAPTAGAALMLCAATAVLLSAQGGPVQSKSPRFASWAEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPAVAVALPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 6 - protein, 406 aa, synthetic construct
MSGAPTAGAALMLCAATAVLLSAQGGPVQSKSPRFASWDKMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPAGAAGLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 7 - protein, 406 aa, synthetic construct
MSGAPTAGAALMLCAATAVLLSAQGGPVQSKSPRFASWDKMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPAGSGSLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 8 - protein, 406 aa, synthetic construct
MSGAPTAGAALMLCAATAVLLSAQGGPVQSKSPRFASWDKMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPAAAVVLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 9 - protein, 406 aa, synthetic construct
MSGAPTAGAALMLCAATAVLLSAQGGPVQSKSPRFASWDAMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPAGVVALPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 10 - protein, 406 aa, synthetic construct
MSGAPTAGAALMLCAATAVLLSAQGGPVQSKSPRFASWKEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPASGGGLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS